(19) United States
(12) Patent Application Publication
(10) Pub. No.: US 2017/0363556 A1
(43) Pub. Date: Dec. 21, 2017

(54) GAS SENSOR

(71) Applicant: FUJI ELECTRIC CO., LTD., Kanagawa (JP)

(72) Inventors: Tokumi NAGASE, Tochigi (JP); Naoyoshi MURATA, Tokyo (JP); Takuya SUZUKI, Tokyo (JP)

(21) Appl. No.: 15/493,587

(22) Filed: Apr. 21, 2017

(30) Foreign Application Priority Data
Jun. 15, 2016 (JP) 2016-119265

(51) Int. Cl. G01N 27/12; G01N 33/00

(52) U.S. Cl. CPC G01N 27/125; G01N 27/128; G01N 33/0047

(57) ABSTRACT

A gas sensor includes an adsorption layer configured to cause gas to adsorb thereon, the gas including a target gas and a non-target gas, and a sensor layer covered with the adsorption layer and having an electric characteristic thereof changing in response to density of the target gas passing through the adsorption layer, wherein the adsorption layer is made of a material whose primary component is a metal oxide having gold particles attached thereto.

### BACKGROUND OF THE INVENTION

### 1. Field of the Invention

The disclosures herein relate to a gas sensor.

### 2. Description of the Related Art

A gas sensor known in the art is capable of discriminating between gas components generated at the time of fire and carbon monoxide generated at the time of incomplete combustion or between a gas such as methane detected at the time of leakage of utility gas and miscellaneous gasses such as ethanol (see Patent Document 1, for example).

However, such a gas detector may fail to accurately detect a target gas in an atmosphere which includes both the target gas and other gases that are not a target for detection, as in the case of detecting acetone in an atmosphere which includes acetone and ethanol.

According to one aspect, there may be a need for a gas sensor that is capable of accurately detecting a target gas.

### RELATED-ART DOCUMENTS

### Patent Document

[Patent Document 1] Japanese Patent Application Publication No. 2001-175969

### SUMMARY OF THE INVENTION

According to one embodiment, a gas sensor includes an adsorption layer configured to cause gas to adsorb thereon, the gas including a target gas and a non-target gas, and a sensor layer covered with the adsorption layer and having an electric characteristic thereof changing in response to density of the target gas passing through the adsorption layer, wherein the adsorption layer is made of a material whose primary component is a metal oxide having gold particles attached thereto.

According to at least one embodiment, a disclosed gas sensor is capable of accurately detecting a target gas.

### BRIEF DESCRIPTION OF THE DRAWINGS

FIG. 1 is a schematic cross-sectional view of a gas sensor according to an embodiment;

FIG. 2 is a drawing illustrating the sensitivity characteristics of a gas sensor of the first example;

FIG. 3 is a drawing illustrating the sensitivity characteristics of a gas sensor of the second example;

FIG. 4 is a drawing illustrating the sensitivity characteristics of a gas sensor of the third example;

FIG. 5 is a drawing illustrating the sensitivity characteristics of a gas sensor of the first comparative example; and

FIG. 6 is a drawing illustrating the sensitivity characteristics of a gas sensor of the second comparative example.

### DESCRIPTION OF THE PREFERRED EMBODIMENTS

In the following, embodiments of the present invention will be described with reference to the accompanying drawings. In the specification and drawings, elements having substantially the same functions or configurations are referred to by the same numerals, and a duplicate description thereof will be omitted.

A gas sensor according to an embodiment is capable of accurately detecting a gas that is a target for detection even when the atmosphere includes such a target gas and a gas that is not a target for detection.

In the following, an example will be described by referring to a semiconductor gas sensor capable of accurately detecting acetone in an atmosphere that includes acetone serving as an example of a target gas and ethanol serving as an example of a non-target gas. A gas sensor capable of accurately detecting acetone may preferably be used as a breath measuring device configured to detect acetone in the breath for use in health condition management. The target gas may be another gas that is not acetone, and the non-target gas may be another gas that is not ethanol.

### <Gas Sensor>

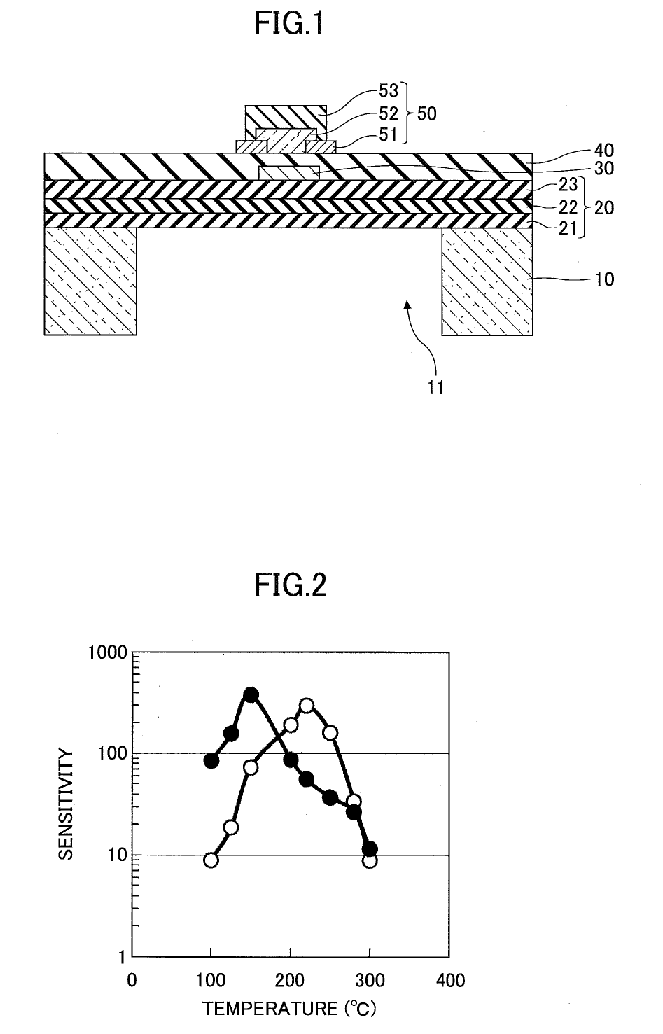

A gas sensor of the embodiment will be described by referring to FIG. 1. FIG. 1 is a schematic cross-sectional view of a gas sensor according to the embodiment.

The gas sensor illustrated in FIG. 1, which is a semiconductor gas sensor having a diaphragm structure, includes a substrate 10, a thermally insulating support layer 20, a heater layer 30, an insulating layer 40, and a gas detection layer 50. The heater layer 30 is electrically coupled to a drive and process unit (not shown), which drives the heater layer 30 serving to operate as a heater. The gas detection layer 50 is electrically coupled to the drive and process unit (not shown), which reads the electric characteristics of a sensor layer 52 of the gas detection layer 50. The sensor layer 52 will be described later.

The substrate 10 is a member made of semiconductor material such as silicon (Si), for example. The substrate 10 has a penetrating hole 11 penetrating the substrate 10 at the center thereof.

The thermally insulating support layer 20, which is formed on the substrate 10, has a diaphragm structure. The thermally insulating support layer 20 includes a thermally oxidized film 21, a support film 22, and a thermally insulating film 23.

The thermally oxidized film 21 is formed on the substrate 10 and implemented as a thermally oxidized SiO₂ film, for example. The thermally oxidized film 21 serves to reduce thermal capacity such as to prevent the heat generated by the heater layer 30 from propagating to the substrate 10. The thermally oxidized film 21 also has high etching electivity relative to the substrate 10.

The support film 22 is formed on the thermally oxidized film 21 and implemented as a CVD-Si₃N₄ film, for example. The support film 22 serves as a support film for the diaphragm structure.

The thermally insulating film 23 is formed on the support film 22 and implemented as a CVD-SiO₂ film, for example. The thermally insulating film 23 exhibits strong adhesion to the heater layer 30, and provides electrical insulation between the substrate 10 and the heater layer 30.

The heater layer 30 is formed on the thermally insulating support layer 20 at the center of the position of the penetrating hole 11 in a plan view. With this arrangement, the heater layer 30 is thermally isolated from the substrate 10. The heater layer 30, which is coupled to a power supply (not shown), generates heat upon receiving electric power from the power supply to heat the gas detection layer 50. More specifically, the heater layer 30 heats an adsorption layer 53 to the temperature (e.g., 200 to 250 degrees Celsius) that oxidizes ethanol such as to reduce ethanol reaching the sensor layer 52. The adsorption layer 53 will be described later. With the thermal isolation between the heater layer 30 and the substrate 10, virtually no heat generated by the heater layer 30 dissipates into the surroundings. This arrangement effectively serves to increase the temperature of the gas detection layer 50. The heater layer 30 is implemented as an alloy film of platinum (Pt) and tungsten (W), which will hereinafter be referred to as a “Pt—W film”.

The insulating layer 40 is formed on the thermally insulating support layer 20 to cover the heater layer 30 and implemented as a sputtered SiO₂ film, for example. The insulating layer 40 provides electrical insulation between the heater layer 30 and the gas detection layer 50. The insulating layer exhibits strong adhesion to the gas detection layer 50.

The gas detection layer 50 is formed on the insulating layer 40 at the center of the position of the penetrating hole 11 in a plan view. Namely, the gas detection layer 50 is formed on the insulating layer 40 at the same position as the position of the heater layer 30 in the plan view. The gas detection layer 50 includes an electrode layer 51, the sensor layer 52, and the adsorption layer 53. In the case of adhesion being not sufficient between the insulating layer 40 and the electrode layer 51, a bond layer made of a tantalum (Ta) film or a titanium (Ti) film, for example, may be disposed between the insulating layer 40 and the electrode layer 51.

The electrode layer 51, which is a pair of metal films formed on the insulating layer 40 at the opposite ends of the sensor layer 52 in a plan view, is implemented as a Pt film or a gold (Au) film, for example.

The sensor layer 52 is formed on the insulating layer 40 in contact with the pair of films of the electrode layer 51. The sensor layer 52 is made of a metal oxide such as SnO₂. The sensor layer 52 has the electric characteristics such as a resistance value thereof changing in response to the density of acetone passing through the adsorption layer 53. The sensor layer 52 may be made of a material different from SnO₂, which may be a material containing a metal-oxide semiconductor as a primary component such as In₂O₃, WO₃, ZnO, TiO₂, or the like. In the disclosures herein, the term “primary component” means that the proportion of the component in the material is 50% or more.

The adsorption layer 53 is formed on the electrode layer 51 and the sensor layer 52 such as to cover the surface of the sensor layer 52 for adsorption of gasses inclusive of acetone and ethanol. The adsorption layer 53 is made of Al₂O₃ having gold (Au) particles attached thereto (Au—Al₂O₃), for example. The Au particles of the adsorption layer 53 serve as a catalyst, which facilitates oxidation (i.e., combustion) of ethanol while bringing about virtually no oxidation of acetone at the temperature range of 200 to 250 degrees Celsius, for example. With this arrangement, the adsorption layer 53 enables the removal of ethanol through selective oxidation relative to acetone. In this manner, the adsorption layer 53 reduces ethanol reaching the sensor layer 52 while selectively allowing acetone to reach the sensor layer 52. As a result, the sensor layer 52 can detect acetone separated from ethanol, thereby enabling an accurate detection of acetone. The adsorption layer 53 may be made of a material different from Au—Al₂O₃, which may be a material containing as a primary component a metal oxide insulating material such as ZrO₂, CeO₂, SiO₂, Cr₂O₃, Fe₂O₃, Ni₂O₃, or the like having Au particles attached thereto (or contained therein, or mixed therewith). The average diameter of Au particles may preferably be 5 nm or smaller from the viewpoint of achieving high catalytic activation.

As was described above, the gas sensor of the embodiment has an adsorption layer made of a material whose primary component is a metal oxide having Au particles attached thereto, thereby being capable of accurately detecting acetone.

### <Method of Manufacturing Gas Sensor>

A description will be given of a method of making the gas sensor of the embodiment.

First, the thermally insulating support layer 20 is formed on the substrate 10. Specifically, the substrate 10 is subjected to thermal oxidation, so that the thermally oxidized film 21 is formed in the surface of the substrate 10. Next, silicon nitride (Si₃N₄) is deposited on the thermally oxidized film 21 by a plasma CVD process, which forms the support film 22. Then, silicon oxide (SiO₂) is deposited on the support film 22 by a plasma CVD process, which forms the thermally insulating film 23.

The heater layer 30 is then formed on the thermally insulating support layer 20. To be more specific, a Pt—W film is formed by sputtering on the thermally insulating film 23 at the center of the position of the penetrating hole 11 in a plan view, thereby forming the heater layer 30. In so doing, a metal mask or the like may be used that has an opening at the position where the Pt—W film is to be formed.

Subsequently, the insulating layer 40 is then formed on the thermally insulating support layer 20 to cover the heater layer 30. More specifically, SiO₂ is deposited by sputtering on the thermally insulating support layer 20 to cover the surface of the heater layer 30, thereby forming the insulating layer 40.

The gas detection layer 50 is then formed on the insulating layer 40. Specifically, Pt is deposited by sputtering on the insulating layer 40 to form a pair of films as the electrode layer 51. SnO₂ is then deposited by sputtering on the insulating layer 40 such as to be in contact with the pair of films of the electrode layer 51, thereby forming the sensor layer 52. A paste made by adding together, in equal weight proportion, organic solvent and γ-alumina with an Au-particle additive and by further adding silica-sol binder thereto is then pasted by screen printing on the electrode layer 51 and the sensor layer 52 in such a manner to cover the sensor layer 52, followed by calcination to form the adsorption layer 53. The adsorption layer 53 may be formed as a layer stacked on the surface of the sensor layer 52, or may be formed to cover the electrode layer 51 and the surface of the sensor layer 52.

The penetrating hole 11 is then formed through the substrate 10. More specifically, the substrate 10 is etched from the back surface (from the surface on which the gas detection layer 50 is not formed) by plasma etching to remove Si in the area inclusive of the position of the gas detection layer 50 in a plan view, thereby forming the penetrating hole 11. The high etching selectivity of the thermally oxidized film 21 relative to the substrate 10 allows only the substrate 10 to be etched while avoiding etching the thermally oxidized film 21. Namely, the thermally oxidized film 21 serves as an etching stop film when etching the substrate 10.

The above-described processes produce a gas sensor having the diaphragm structure as illustrated in FIG. 1. This is only an example of a method of making a gas sensor, and is not intended to be limiting. For example, the electrode layer 51 and the sensor layer 52 may be formed, followed by forming the penetrating hole 11 through the substrate 10, and then forming the adsorption layer 53 to cover the sensor layer 52.

### EXAMPLE

In the following, a specific example of a gas sensor will be described.

### First Example

An Si substrate serving as the substrate was subjected to thermal oxidation, thereby forming a thermally oxidized SiO₂ film serving as the thermally oxidized film 21 on the Si substrate. A CVD-Si₃N₄ film serving as the support film 22 and a CVD-SiO₂ film serving as the thermally insulating film 23 were formed in this order on the thermally oxidized SiO₂ film by a plasma CVD process.

Subsequently, a Pt—W film serving as the heater layer 30 was formed on the CVD-SiO₂ film by a sputtering process performed by an RF magnetron sputtering apparatus. An SiO₂ film serving as the insulating layer 40 was then formed on the Pt—W film by a sputtering process performed by the RF magnetron sputtering apparatus.

After this, a sputtering process was performed by the RF magnetron sputtering apparatus to form a Pt film having a film thickness of 200 nm serving as the electrode layer 51 on the SiO₂ film under the conditions of an Ar gas pressure of 1 Pa, a substrate temperature of 300 degrees Celsius, and an RF power of 2 W/cm².

A reactive sputtering process was thereafter performed by the RF magnetron sputtering apparatus to form an SiO₂ film having a film thickness of 400 nm serving as the sensor layer 52 on the SiO₂ film under the conditions of an Ar+O₂ gas pressure of 2 Pa, a substrate temperature of 150 to 300 degrees Celsius, and an RF power of 2 W/cm². In so doing, SnO₂ containing 0.1 wt % antimony (Sb) was used as a target material.

A paste was then produced by adding together, in equal weight, organic solvent and γ-Al₂O₃ (with an average particle diameter of 1 to 2 micrometers) with an additive of 0.7 wt % Au particles having an average diameter of 3.5 nm and by further adding 5 wt % to 20 wt % of silica-sol binder. The paste was then applied by screen printing on the Pt film and the SnO₂ film to form a circular shape with a thickness of approximately 30 micrometers and a diameter of 250 micrometers, followed by calcination at 300 degrees Celsius for 12 hours to form an Au—Al₂O₃ film serving as the adsorption layer 53.

Plasma etching was then performed to etch the Si substrate from the back surface to remove Si in the area inclusive of the position where the Pt—W film, the Pt film, the SnO₂ film, and the Au—Al₂O₃ film were formed, thereby forming the penetrating hole 11.

The above-described processes produced a gas sensor having the diaphragm structure as illustrated in FIG. 1.

### Second Example

In the second example, a gas sensor was produced in the same manner as in the first example, except that different method and materials than in the first example were used to form the adsorption layer. Specifically, a paste was then produced by adding together, in equal weight, organic solvent and CeO₂ (with an average particle diameter of 2 to micrometers) with an additive of 0.6 wt % Au particles having an average diameter of 3 nm and by further adding 5 wt % to 20 wt % of silica-sol binder. The paste was then applied by screen printing on the Pt film and the SnO₂ film to form a circular shape with a thickness of approximately 30 micrometers and a diameter of 250 micrometers, followed by calcination at 300 degrees Celsius for 12 hours. With these processes, a CeO₂ (Au—CeO₂) film having Au particles attached thereto was formed to serve as the adsorption layer.

### Third Example

In the third example, a gas sensor was produced in the same manner as in the first example, except that different method and materials than in the first example were used to form the adsorption layer. Specifically, a paste was then produced by adding together, in equal weight, organic solvent and ZrO₂ (with an average particle diameter of 2 to 3 micrometers) with an additive of 0.6 wt % Au particles having an average diameter of 3 nm and by further adding 5 wt % to 20 wt % of silica-sol binder. The paste was then applied by screen printing on the Pt film and the SnO₂ film to form a circular shape with a thickness of approximately 30 micrometers and a diameter of 250 micrometers, followed by calcination at 300 degrees Celsius for 12 hours. With these processes, a ZrO₂ (Au—ZrO₂) film having Au particles attached thereto was formed to serve as the adsorption layer.

### First Comparative Example

In the first comparative example, a gas sensor was produced in the same manner as in the first example, except that different method and materials than in the first example were used to form the adsorption layer. Specifically, a paste was then produced by adding together, in equal weight, diethylene glycol monoethyl ether and γ-Al₂O₃ (with an average particle diameter of 2 to 3 micrometers) with an additive of 7.0 wt % palladium (Pd) and by further adding 5 wt % to 20 wt % of silica-sol binder. The paste was then applied by screen printing on the Pt film and the SnO₂ film to form a circular shape with a thickness of approximately 30 micrometers and a diameter of 250 micrometers, followed by calcination at 500 degrees Celsius for 12 hours. With these processes, an Al₂O₃ (Pd—Al₂O₃) film with Pd particles attached thereof was formed to serve as the adsorption layer.

### Second Comparative Example

In the second comparative example, a gas sensor was produced in the same manner as in the first example, except that different method and materials than in the first example were used to form the adsorption layer. Specifically, a paste was then produced by adding together, in equal weight, diethylene glycol monoethyl ether and γ-Al₂O₃ (with an average particle diameter of 2 to 3 micrometers) without any catalyst and by further adding 5 wt % to 20 wt % of silica-sol binder. The paste was then applied by screen printing on the Pt film and the SnO₂ film to have a thickness of approximately 30 micrometers and a diameter of 250 micrometers, followed by calcination at 500 degrees Celsius for hours. With these processes, an Al₂O₃ film with no catalysts was formed to serve as the adsorption layer.

### <Evaluation>

The gas sensors produced in the first through third examples and the first and second comparative examples were subjected to intermittent activation having a 30-second period with 1-second activation while the temperature of the gas sensor was set at 300 degrees Celsius, followed by changing the sensor temperature, and then measuring the resistance value of the gas sensor (i.e., the sensor layer 52) in the state of stable resistance. Gas sensitivity was derived from the measured resistance value. Gas sensitivity is calculated as Rair/Rgas where Rair represents the resistance value of the gas sensor in a clean air atmosphere that includes neither acetone nor ethanol, and Rgas represents the resistance value of the gas sensor in a gas atmosphere that includes a predetermined density of acetone or ethanol. Gas sensitivity being equal to 1 means that gas sensitivity is completely nonexistent. A difference between the sensitivity of acetone and the sensitivity of ethanol may be obtained by using the difference in sensitivity at a predetermined temperature (i.e. at a fixed temperature point), or may be obtained by using a difference between the averages of respective sensitivities obtained over a plurality of points in a predetermined temperature range. It suffices to use an evaluation method that brings about a great sensitivity difference.

FIG. 2 is a drawing illustrating the sensitivity characteristics of the gas sensor of the first example. FIG. 2 illustrates the temperature dependency of sensitivity in a gas atmosphere that includes 1 ppm of acetone and the temperature dependency of sensitivity in a gas atmosphere that includes 1 ppm of ethanol. In FIG. 2, the horizontal axis represents the temperature of the gas sensor in Celsius, and the vertical axis represents the sensitivity of the gas sensor. Open circles represent the temperature dependency of sensitivity with respect to acetone, and solid circles represent the temperature dependency of sensitivity with respect to ethanol.

As illustrated in FIG. 2, sensitivity for acetone and sensitivity for ethanol exhibit different tendencies when the temperature of the gas sensor is in the range of 200 to 250 degrees Celsius, such that sensitivity for acetone is higher than sensitivity for ethanol. This appears to be because γ-Al₂O₃ with an additive of Au particles having an average diameter of 5 nm or smaller serves to oxidize ethanol and to oxidize virtually no acetone in the range of 200 to 250 degrees Celsius. Accordingly, the gas sensor of the first example utilizes a difference between sensitivity for acetone and sensitivity for ethanol to discriminate between acetone and ethanol for successful detection.

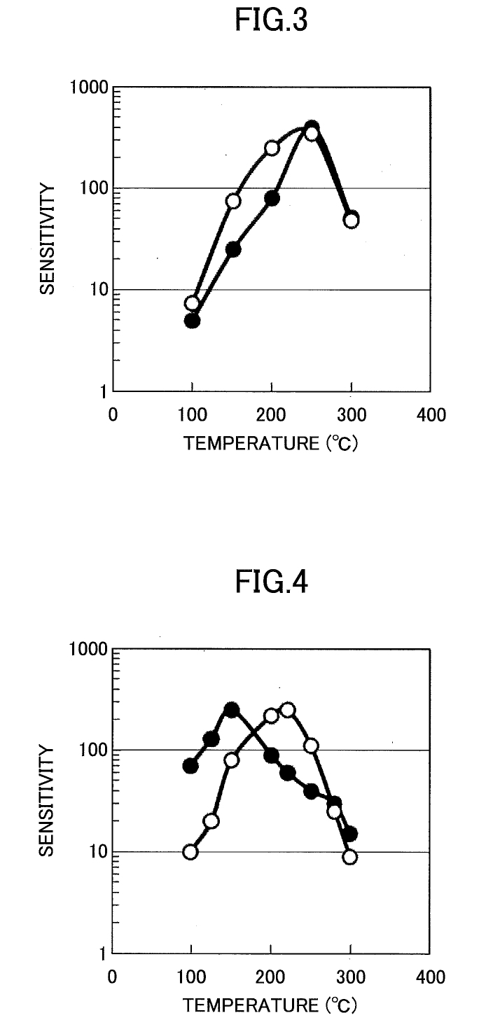

FIG. 3 is a drawing illustrating the sensitivity characteristics of the gas sensor of the second example. FIG. 3 illustrates the temperature dependency of sensitivity in a gas atmosphere that includes 1 ppm of acetone and the temperature dependency of sensitivity in a gas atmosphere that includes 1 ppm of ethanol. In FIG. 3, the horizontal axis represents the temperature of the gas sensor in Celsius, and the vertical axis represents the sensitivity of the gas sensor. Open circles represent the temperature dependency of sensitivity with respect to acetone, and solid circles represent the temperature dependency of sensitivity with respect to ethanol.

As illustrated in FIG. 3, sensitivity for acetone and sensitivity for ethanol exhibit different tendencies when the temperature of the gas sensor is in the range of 100 to 200 degrees Celsius, such that sensitivity for acetone is higher than sensitivity for ethanol. This appears to be because CeO₂ with an additive of Au particles having an average diameter of 5 nm or smaller serves to oxidize ethanol and to oxidize virtually no acetone in the range of 100 to 200 degrees Celsius. Accordingly, the gas sensor of the second example utilizes a difference between sensitivity for acetone and sensitivity for ethanol to discriminate between acetone and ethanol for successful detection.

FIG. 4 is a drawing illustrating the sensitivity characteristics of the gas sensor of the third example. FIG. 4 illustrates the temperature dependency of sensitivity in a gas atmosphere that includes 1 ppm of acetone and the temperature dependency of sensitivity in a gas atmosphere that includes 1 ppm of ethanol. In FIG. 4, the horizontal axis represents the temperature of the gas sensor in Celsius, and the vertical axis represents the sensitivity of the gas sensor. Open circles represent the temperature dependency of sensitivity with respect to acetone, and solid circles represent the temperature dependency of sensitivity with respect to ethanol.

As illustrated in FIG. 4, sensitivity for acetone and sensitivity for ethanol exhibit different tendencies when the temperature of the gas sensor is in the range of 200 to 250 degrees Celsius, such that sensitivity for acetone is higher than sensitivity for ethanol. This appears to be because ZrO₂ with an additive of Au particles having an average diameter of 5 nm or smaller serves to oxidize ethanol and to oxidize virtually no acetone in the range of 200 to 250 degrees Celsius. Accordingly, the gas sensor of the third example utilizes a difference between sensitivity for acetone and sensitivity for ethanol to discriminate between acetone and ethanol for successful detection.

FIG. 5 is a drawing illustrating the sensitivity characteristics of the gas sensor of the first comparative example. FIG. 5 illustrates the temperature dependency of sensitivity in a gas atmosphere that includes 1 ppm of acetone and the temperature dependency of sensitivity in a gas atmosphere that includes 1 ppm of ethanol. In FIG. 5, the horizontal axis represents the temperature of the gas sensor in Celsius, and the vertical axis represents the sensitivity of the gas sensor. Open circles represent the temperature dependency of sensitivity with respect to acetone, and solid circles represent the temperature dependency of sensitivity with respect to ethanol.

As illustrated in FIG. 5, sensitivity for acetone and sensitivity for ethanol exhibit substantially the same tendencies when the temperature of the gas sensor falls within the range (i.e., 100 to 300 degrees Celsius) in which sensitivity for acetone is high. This appears to be because virtually no oxidation of acetone and virtually no oxidation of ethanol occur in the temperature range in which sensitivity for acetone is high, despite the fact that Pd is added in the adsorption layer as a catalyst. Because of this, the gas sensor of the first comparative example is incapable of discriminate between acetone and ethanol for detection.

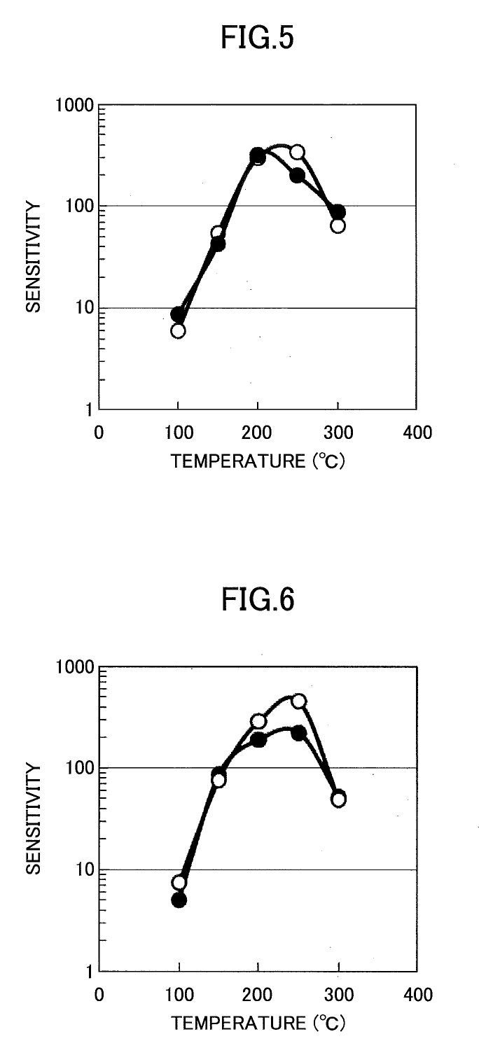

FIG. 6 is a drawing illustrating the sensitivity characteristics of the gas sensor of the second comparative example. FIG. 6 illustrates the temperature dependency of sensitivity in a gas atmosphere that includes 1 ppm of acetone and the temperature dependency of sensitivity in a gas atmosphere that includes 1 ppm of ethanol. In FIG. 6, the horizontal axis represents the temperature of the gas sensor in Celsius, and the vertical axis represents the sensitivity of the gas sensor. Open circles represent the temperature dependency of sensitivity with respect to acetone, and solid circles represent the temperature dependency of sensitivity with respect to ethanol.

As illustrated in FIG. 6, sensitivity for acetone and sensitivity for ethanol exhibit substantially the same tendencies when the temperature of the gas sensor falls within the range (i.e., 100 to 300 degrees Celsius) in which sensitivity for acetone is high. This appears to be because the adsorption layer including no catalysts results in virtually no occurrence of either oxidation of acetone or oxidation of ethanol. Because of this, the gas sensor of the second comparative example is incapable of discriminate between acetone and ethanol for detection.

Further, the present invention is not limited to these embodiments, but various variations and modifications may be made without departing from the scope of the present invention.

The present application is based on Japanese priority application No. 2016-119265 filed on Jun. 15, 2016, with the Japanese Patent Office, the entire contents of which are hereby incorporated by reference.

What is claimed is

1. A gas sensor, comprising:
an adsorption layer configured to cause gas to adsorb thereon, the gas including a target gas and a non-target gas; and
a sensor layer covered with the adsorption layer and having an electric characteristic thereof changing in response to density of the target gas passing through the adsorption layer,
wherein the adsorption layer is made of a material whose primary component is a metal oxide having gold particles attached thereto.

2. The gas sensor as claimed in claim 1, further comprising a heater layer configured to heat the adsorption layer to such a temperature that oxidation of non-target gas occurs to reduce the non-target gas reaching the sensor layer.

3. The gas sensor as claimed in claim 1, wherein an average diameter of the gold particles is 5 nm or smaller.

4. The gas sensor as claimed in claim 1, wherein the metal oxide includes at least one of Al₂O₃, ZrO₂,CeO₂, Cr₂O₃, Fe₂O₃, and Ni₂O₃.

5. The gas sensor as claimed in claim 1, wherein the sensor layer is made of a material whose primary component is a metal-oxide semiconductor.

6. The gas sensor as claimed in claim 5, wherein the metal-oxide semiconductor includes at least one of SnO₂, In₂O₃, WO₃, ZnO, and TiO₂.

7. The gas sensor as claimed in claim 1, wherein the target gas is acetone, and the non-target gas is ethanol.
